(19) United States
(12) Patent Application Publication
(10) Pub. No.: US 2024/0016230 A1
(43) Pub. Date: Jan. 18, 2024

(54) AEROSOL PROVISION SYSTEM

(71) Applicant: NICOVENTURES TRADING LIMITED, LONDON (GB)

(72) Inventors: Nicholas ROSSER, London (GB); Connor BRUTON, London (GB); Charanjit NANDRA, London (GB); David RUSHFORTH, London (GB); David BAKER, London (GB); Robert KERSEY, London (GB); Mark CROSIER, London (GB)

(21) Appl. No.: 18/004,984

(22) PCT Filed: Jul. 6, 2021
(86) PCT No.: PCT/GB2021/051716

§ 371 Date: Jan. 10, 2023

(30) Foreign Application Priority Data
Jul. 10, 2020 (GB) 2010610.0

(51) Int. Cl. A24F 40/51; A24F 40/53; A24F 40/65

(52) U.S. Cl. CPC A24F 40/51; A24F 40/53; A24F 40/65; A24F 40/10

(57) ABSTRACT

An aerosol provision system includes an aerosol generator configured to aerosolize an aerosol-generating material. The aerosol provision system also includes a sensor configured to detect an inhalation on the aerosol provision system by a user of the aerosol provision system, and output corresponding inhalation detection signals to control circuitry. The control circuitry is configured to determine an indication of an amount of an ingredient delivered from the aerosol generating material to the user during the inhalation based on the inhalation detection signals, compare the indication of the amount of the ingredient delivered to a threshold, and provide a notification to the user based on the comparison between the indication of the amount of the ingredient delivered to the threshold.

### PRIORITY CLAIM

The present application is a National Phase entry of PCT Application No. PCT/GB2021/051716, filed Jul. 6, 2021, which claims priority from GB Application No. 2010610.0, filed Jul. 10, 2020, each of which hereby fully incorporated herein by reference.

### TECHNICAL FIELD

The present disclosure relates to an aerosol provision system.

### BACKGROUND

Electronic aerosol provision systems such as electronic cigarettes (e-cigarettes) generally contain an aerosol-generating material, such as a reservoir of a source liquid containing a formulation, typically including nicotine, or a solid material such as a tobacco-based product, from which an aerosol is generated for inhalation by a user, for example through heat vaporization. Thus, an aerosol provision system will typically comprise an aerosol generator, e.g. a heating element, arranged to aerosolize a portion of aerosol-generating material to generate an aerosol in an aerosol generation region of an air channel through the aerosol provision system. As a user inhales on the device and electrical power is supplied to the aerosol generator, air is drawn into the device through one or more inlet holes and along the air channel to the aerosol generation region, where the air mixes with the vaporized aerosol generator and forms a condensation aerosol. The air drawn through the aerosol generation region continues along the air channel to a mouthpiece, carrying some of the aerosol with it, and out through the mouthpiece for inhalation by the user.

It is common for aerosol provision systems to comprise a modular assembly, often having two main functional parts, namely an aerosol provision device and disposable/replaceable consumable part. Typically the consumable will comprise the consumable aerosol-generating material and the aerosol generator (heating element), while the aerosol provision device part will comprise longer-life items, such as a rechargeable battery, device control circuitry and user interface features. The aerosol provision device may also be referred to as a reusable part or battery section and the consumable may also be referred to as a disposable part, cartridge or cartomizer.

The aerosol provision device and consumable are mechanically coupled together at an interface for use, for example using a screw thread, bayonet, latched or friction fit fixing. When the aerosol-generating material in a consumable has been exhausted, or the user wishes to switch to a different consumable having a different aerosol-generating material, the consumable may be removed from the aerosol provision device and a replacement consumable may be attached to the device in its place.

A potential drawback for aerosol provision systems is that there is no means to monitor the usage of the aerosol provision system by the user. This may lead to excessive use of the system by the user. Equally, the user may have little understanding of how they are using the system, such as when or how often they use the system.

Various approaches are described herein which seek to help address or mitigate some of the issues discussed above.

### SUMMARY

In accordance with some embodiments described herein, there is provided an aerosol provision system. The aerosol provision system comprises an aerosol generator configured to aerosolize an aerosol-generating material. The aerosol provision system also comprises a sensor configured to detect an inhalation on the aerosol provision system by a user of the aerosol provision system, and output corresponding inhalation detection signals to control circuitry. The control circuitry is configured to determine an indication of an amount of an ingredient delivered from the aerosol generating material to the user during the inhalation based on the inhalation detection signals, compare the indication of the amount of the ingredient delivered to a threshold, and provide a notification to the user based on the comparison between the indication of the amount of the ingredient delivered to the threshold.

The comparison of the indication of the amount of the ingredient delivered to the threshold may be performed based on an indication of the amount of the ingredient delivered from the aerosol-generating material to the user during each inhalation in a predetermined period, for example an hour, 24 hours or a week.

The comparison of the indication of the amount of the ingredient delivered to the threshold may be performed after each inhalation. The comparison of the indication of the amount of the ingredient delivered to the threshold may be performed during each inhalation, for example at a predetermined interval during the inhalation.

The determination of the indication of the amount of the ingredient delivered from the aerosol-generating material to the user may be based on a concentration of the ingredient in the aerosol-generating material. The ingredient may be nicotine, caffeine, taurine, theine, a vitamin, melatonin, or a cannabinoid.

The notification may be provided to the user when the indication of the amount of the ingredient delivered exceeds the threshold.

The notification may be provided to the user based on the comparison between the indication of the amount of the ingredient delivered to a plurality of thresholds.

The control circuitry may be configured to determine default user behavior based on the indication of the amount of the ingredient delivered from the aerosol-generating material to the user. The default user behavior may determined over an inhalation. The control circuitry may be configured to determine a time between the inhalation and a next inhalation based on the inhalation detection signals, and determine default user behavior based on the time between the inhalation and the next inhalation. The default user behavior may be determined over a plurality of inhalations, and a time between each of the plurality of inhalations is less than a predetermined time. The default user behavior may be determined over a rolling time period. The threshold may be determined based on the determined default user behavior.

The notification may be provided on the aerosol provision system and/or an application on a remote device. The notification may be a haptic notification. A parameter of the haptic notification may be adjustable by the user. The notification may be configurable by the user.

In accordance with some embodiments described herein, there is provided a system comprising an aerosol provision system configured to generate aerosol from an aerosol-generating material. The system also comprises a computer configured to determine an indication of an amount of an ingredient delivered from the aerosol-generating material aerosolized by an aerosol generator to the user during the inhalation based on inhalation detection signals received from a sensor of the aerosol provision system, compare the indication of the amount of the ingredient to a threshold, and provide a notification to the user based on the comparison between the indication of the amount of the ingredient delivered to the threshold.

In accordance with some embodiments described herein, there is provided a method of operating an aerosol provision system comprising receiving inhalation detection signals from a sensor configured to detect an inhalation on the aerosol provision system by a user of aerosol provision system, determining an indication of an amount of an ingredient delivered from an aerosol-generating material aerosolized by an aerosol generator to the user during the inhalation based on the inhalation detection signals, comparing the indication of the amount of the ingredient to a threshold, and providing a notification to the user based on the comparison between the indication of the amount of the ingredient delivered to the threshold. There is also provided a computer readable storage medium comprising instructions which, when executed by a processor, performs the above method.

These aspects and other aspects will be apparent from the following detailed description. In this regard, particular sections of the description are not to be read in isolation from other sections.

### BRIEF DESCRIPTION OF DRAWINGS

Embodiments of the disclosure will now be described, by way of example only, with reference to accompanying drawings, in which:

FIGS. 1 and 2 are schematic diagrams of an aerosol provision system.

FIGS. 3A to 3C illustrate graphs of inhalation detection signal output by the sensor against time.

FIG. 4 illustrates a system comprising an aerosol provision system and a computer.

FIG. 5 is a flow chart of a method of operating an aerosol provision system.

### DETAILED DESCRIPTION

Aspects and features of certain examples and embodiments are discussed/described herein. Some aspects and features of certain examples and embodiments may be implemented conventionally and these are not discussed/described in detail in the interests of brevity. It will thus be appreciated that aspects and features of articles and systems discussed herein which are not described in detail may be implemented in accordance with any conventional techniques for implementing such aspects and features.

The present disclosure relates to aerosol provision systems, which may also be referred to as aerosol provision systems, such as e-cigarettes. Throughout the following description the term “e-cigarette” or “electronic cigarette” may sometimes be used, but it will be appreciated this term may be used interchangeably with aerosol provision system and electronic aerosol provision system.

As noted above, aerosol provision systems (e-cigarettes) often comprise a modular assembly including both a reusable part (aerosol provision device) and a replaceable (disposable) cartridge part, referred to as a consumable. Systems conforming to this type of two-part modular configuration may generally be referred to as two-part systems or devices. It is also common for electronic cigarettes to have a generally elongate shape. For the sake of providing a concrete example, certain embodiments of the disclosure described herein comprise this kind of generally elongate two-part system employing disposable cartridges. However, it will be appreciated the underlying principles described herein may equally be adopted for other electronic cigarette configurations, for example modular systems comprising more than two parts, as devices conforming to other overall shapes, for example based on so-called box-mod high performance devices that typically have a more boxy shape.

As described above, the present disclosure relates to (but it not limited to) aerosol provision devices and corresponding aerosol provision systems, such as e-cigarettes and electronic cigarettes.

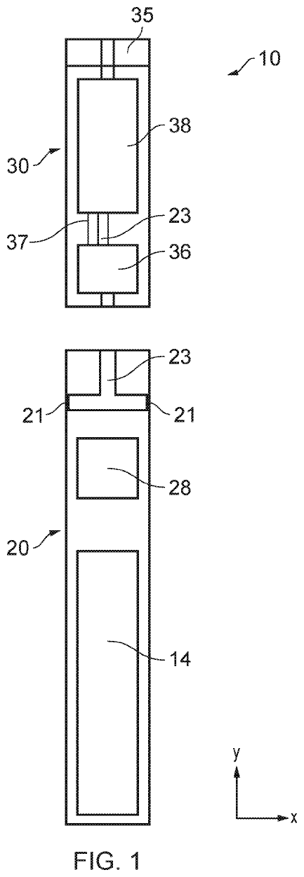

FIG. 1 is a highly schematic diagram (not to scale) of an example aerosol provision system 10, such as an e-cigarette, to which embodiments are applicable. The aerosol provision system has a generally cylindrical shape, extending along a longitudinal or y axis as indicated by the axes (although aspects of the disclosure are applicable to e-cigarettes configured in other shapes and arrangements), and comprises two main components, namely an aerosol provision device 20 and a consumable 30.

The consumable 30 is an article comprising or consisting of aerosol-generating material 38, part or all of which is intended to be consumed during use by a user. A consumable 30 may comprise one or more other components, such as an aerosol-generating material storage area, an aerosol-generating material transfer component 37, an aerosol generation area, a housing, a wrapper, a mouthpiece 35, a filter and/or an aerosol-modifying agent.

A consumable 30 may also comprise an aerosol generator 36, such as a heating element, that emits heat to cause the aerosol-generating material 38 to generate aerosol in use. The aerosol generator 36 may, for example, comprise combustible material, a material heatable by electrical conduction, or a susceptor. It should be noted that it is possible for the aerosol generator 36 to be part of the aerosol provision device 20 and the consumable 30 then may comprise the aerosol-generating material storage area for the aerosol-generating material 38 such that, when the consumable 30 is coupled with the aerosol provision device 20, the aerosol-generating material 38 can be transferred to the aerosol generator 36.

The aerosol-generating material 38 is a material that is capable of generating aerosol, for example when heated, irradiated or energized in any other way. The aerosol-generating material 38 may, for example, be in the form of a solid, liquid or gel which may or may not contain an active substance and/or flavorants. In some embodiments, the aerosol-generating material 38 may comprise an “amorphous solid”, which may alternatively be referred to as a “monolithic solid” (i.e. non-fibrous). In some embodiments, the amorphous solid may be a dried gel. The amorphous solid is a solid material that may retain some fluid, such as liquid, within it. In some embodiments, the aerosol-generating material may for example comprise from about 50 wt %, 60 wt % or 70 wt % of amorphous solid, to about 90 wt %, 95 wt % or 100 wt % of amorphous solid.

The aerosol-generating material 38 comprises one or more ingredients, such as one or more active substances and/or flavorants, one or more aerosol-former materials, and optionally one or more other functional materials such as pH regulators, coloring agents, preservatives, binders, fillers, stabilizers, and/or antioxidants.

The active substance as used herein may be a physiologically active material, which is a material intended to achieve or enhance a physiological response. The active substance may for example be selected from nutraceuticals, nootropics, psychoactives. The active substance may be naturally occurring or synthetically obtained. The active substance may comprise for example nicotine, caffeine, taurine, theine, vitamins such as B6 or B12 or C, melatonin, cannabinoids, or constituents, derivatives, or combinations thereof. The active substance may comprise one or more constituents, derivatives or extracts of tobacco, cannabis or another botanical.

In some embodiments, the active substance comprises nicotine. In some embodiments, the active substance comprises caffeine, melatonin or vitamin B12.

The aerosol provision device 20 includes a power source 14, such as a battery, configured to supply electrical power to the aerosol generator 36. The power source 14 in this example is rechargeable and may be of a conventional type, for example of the kind normally used in electronic cigarettes and other applications requiring provision of relatively high currents over relatively short periods. The battery 14 may be recharged through the charging port (not illustrated), which may, for example, comprise a USB connector.

The aerosol provision device 20 includes control circuitry 28 configured to monitor and/or control the operation of the aerosol provision system 10 and provide conventional operating functions in line with the established techniques for controlling aerosol provision systems such as electronic cigarettes. The control circuitry (processor circuitry) 28 may be considered to logically comprise various sub-units/circuitry elements associated with different aspects of the electronic cigarette's operation. For example, depending on the functionality provided in different implementations, the control circuitry 28 may comprises power source control circuitry for controlling the supply of electrical power from the power source 14 to the aerosol generator 36, user programming circuitry for establishing configuration settings (e.g. user-defined power settings) in response to user input, as well as other functional units/circuitry associated functionality in accordance with the principles described herein and conventional operating aspects of electronic cigarettes. It will be appreciated the functionality of the control circuitry 28 can be provided in various different ways, for example using one or more suitably programmed programmable computer(s) and/or one or more suitably configured application-specific integrated circuit(s)/circuitry/chip(s)/chipset(s) configured to provide the desired functionality.

The aerosol provision device 20 illustrated in FIG. 1 includes one or more air inlets 21. In use, as a user inhales on the mouthpiece 35, air is drawn into the aerosol provision device 20 through the air inlets 21 and along an air channel 23 to the aerosol generator 36, where the air mixes with the vaporized aerosol-generating material 38 and forms a condensation aerosol. The air drawn through the aerosol generator 36 continues along the air channel 23 to a mouthpiece 35, carrying some of the aerosol with it, and out through the mouthpiece 35 for inhalation by the user. It will be appreciated that the one or more air inlets may be provided on the consumable 30 such that the air channel 23 is entirely contained within the consumable 30, or the aerosol provision device 20 and the consumable 30 may each comprise at least one air inlet 21 and a portion of the air channel 23.

By way of a concrete example, the consumable 30 comprises a housing (formed, e.g. from a plastics material), a reservoir formed within the housing for containing the aerosol-generating material 38 (which in this example may be a liquid which may or may not contain nicotine), an aerosol-generating material transfer component 37 (which in this example is a wick formed of e.g., glass or cotton fibers, or a ceramic material configured to transport the liquid from the reservoir using capillary action), an aerosol generating area, and a mouthpiece 35. Although not shown, a filter and/or aerosol modifying agent (such as a flavor imparting material) may be located in, or in proximity to, the mouthpiece 35. The consumable of this example comprises a heater element formed from an electrically resistive material (such as NiCr8020) spirally wrapped around the aerosol-generating material transfer component 37, and located in the air channel 23. The area around the heating element and wick combination is the aerosol generating area of the consumable 30. The consumable comprises suitable electrical contacts for coupling to electrical contacts provided on the aerosol provision device 20, such that electrical power may be supplied directly to the heater element.

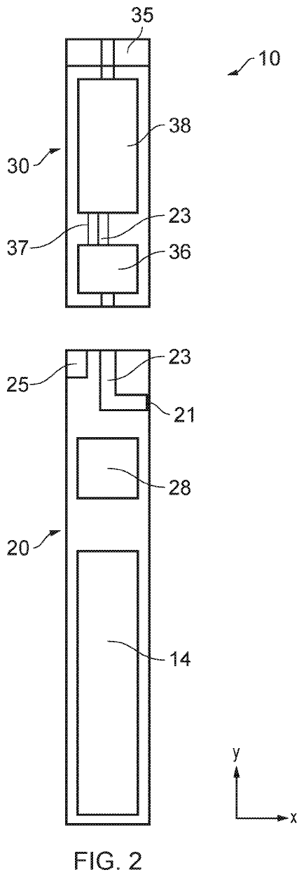

FIG. 2 is a schematic diagram of a further example of an aerosol provision system 10, where the same reference signs have been used for like elements between the aerosol provision system 10 illustrated in FIG. 1 and the aerosol provision system 10 illustrated in FIG. 2.

The aerosol provision system 10 in FIG. 2 comprises a sensor 25 configured to detect an inhalation on the aerosol provision system 10 by a user of the aerosol provision system 10. For example, the sensor 25 may be a flow sensor, a microphone, a pressure sensor, light sensor, touch sensor, accelerometer, gyroscope, or any other type of sensor suitable for directly or indirectly detecting or inferring an inhalation on the aerosol provision system 10 by a user of the aerosol provision system 10. Although the sensor 25 illustrated in FIG. 2 is part of the aerosol provision device 20, this is not essential. In other embodiments the sensor 25 may be part of the consumable 30.

The sensor 25 may be configured to detect an inhalation based on the flow of air into one or more of the air inlets 21, or in the air channel 23 through the aerosol provision system 10. Alternative, the sensor may include a pressure sensor or light sensor on the mouthpiece 35 configured to detect when the user's lips are placed around the mouthpiece 35, or a pressure sensor or light sensor located on the aerosol provision device 20 to detect when the user places their hand around the aerosol provision device 20.

In some examples there is more than one sensor 25. For example, there may be a sensor 25 located proximate to an air inlet and a sensor 25 proximate to a portion of the air channel 23, the aerosol generator 36 and/or the mouthpiece 35 as described above. Accordingly, each sensor is configured to detect an inhalation on the aerosol provision system 10. Where there is more than one sensor 25, this can comprise more than one type of sensor, and/or multiple sensors of the same type.

In response to detecting an inhalation on the aerosol provision system 10 by a user of the system, the sensor 25 is configured to output corresponding inhalation detection signals to the control circuitry 28. In some embodiments, the sensor 25 is configured to continuously output inhalation detection signals, or to output inhalation detection signals periodically, such as every 0.01 seconds, every 0.1 seconds, or every 1 second. If the sensor 25 outputs inhalation detection signals periodically, then in some implementations the period between the output of subsequent inhalation detection signals may be set equal to or less than the average, or a typical length of an user inhalation (e.g. between 2 to 5 seconds) so as to ensure that an inhalation is not missed. In each case, the inhalation detection signals change when an inhalation on the system is detected by the sensor 25. For example, the inhalation detection signals could be a binary indication of whether an inhalation on the system is detected or not, for example a “1” to indicate an inhalation has been detected and a “0” to indicate that an inhalation has not been detected. Alternatively, the inhalation detection signals could correspond to an inhalation level or strength detected by the sensor 25. In other words, the inhalation detection signals could provide an indication of the draw strength detected by the sensor 25. For example, if the sensor 25 is a microphone or a flow sensor, the inhalation detection signals could provide an indication of the air speed or mass flow through the aerosol provision system 10, thereby providing an indication of the magnitude or strength of the inhalation taken by the user. In some embodiments, the inhalation detection signals correspond to the signals detected by the sensor 25. In other words, the inhalation detection signals represent the raw output from the sensor 25 without any filtering or processing applied by the sensor 25.

The inhalation detection signals could be set to 0 when an inhalation has not been detected by the sensor 25 and correspond to the inhalation level or strength detected by the sensor 25 when an inhalation has been detected. In some embodiments, the sensor 25 is configured to only output inhalation detection signals when an inhalation has been detected. In other words, the sensor 25 is configured output inhalation detection in response to detecting an inhalation on the system and the sensor 25 is configured to stop outputting inhalation detection signals when the inhalation is no longer detected by the sensor 25.

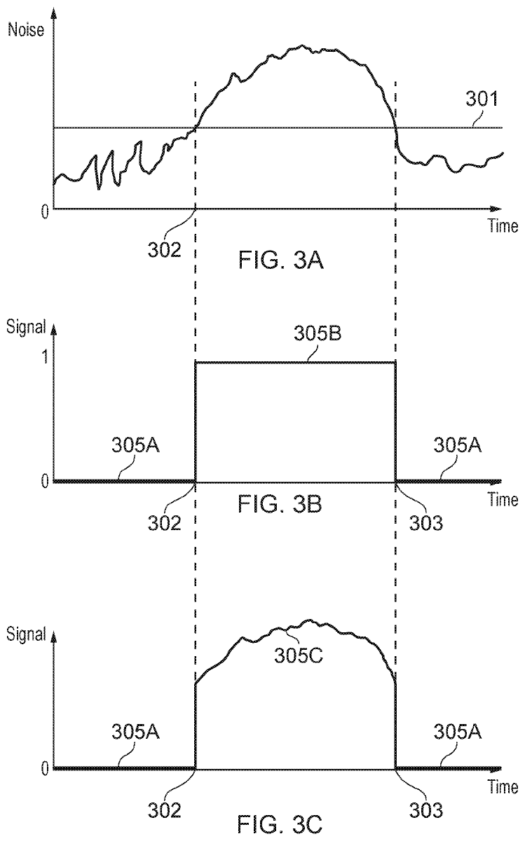

FIGS. 3A to 3C illustrate graphs of inhalation detection signal output by the sensor 25 against time. In the example illustrated in FIG. 3A, the sensor 25 outputs inhalation detection signals continuously, and an inhalation being detected by the sensor 25 corresponds to the period when the inhalation detection signals are greater than a detection threshold 301. In the example illustrated in FIG. 3B, until time point 302, which corresponds to the time point at which the sensor 25 detects an inhalation by the user on the aerosol provision system 10, the inhalation detection signal 305A output by the sensor 25 is “0”. This may either represent no inhalation detection signal being output, or an inhalation detection signal being output with a value equal to “0”. Between time point 301 and 302, which corresponds to the times during which the sensor 25 detects an inhalation by the user on the aerosol provision system 10, the inhalation detection signal 305B output by the sensor 25 is “1”. In other words, the sensor 25 outputs an inhalation detection signal to indicate that an inhalation is detected. After time point 303, the inhalation detection signal 305A output by the sensor 25 is “0”, indicating that the sensor 25 no longer detects an inhalation. As set out above, this may either represent no inhalation detection signal being output, or an inhalation detection signal being output with a value equal to “0”. In the example illustrated in FIG. 3C the inhalation detection signals 305A are set to “0” when an inhalation is not detected by the sensor 25, and the inhalation detection signal 305C corresponds to the signal recorded by the sensor 25 when the sensor 25 detects an inhalation.

As described above, the sensor 25 is configured to output the inhalation detection signals to the control circuitry 28. In response to receiving the inhalation detection signals, the control circuitry 28 is configured to determine an indication of an amount of an ingredient delivered from the aerosol-generating material to the user during the inhalation based on the inhalation detection signals. As it will be appreciated, the amount of aerosol, and by extension the amount of aerosol-generating material 38, delivered to the user during an inhalation will vary based on various factors, such as the duration of the inhalation, the type of aerosol-generating material 38 aerosolized, the temperature of aerosol generator 36, the amount of electrical power delivered to the aerosol generator 36 and the speed or mass flow of the air through the aerosol provision system 10. As described above, the aerosol-generating material 38 comprises one or more ingredients. Accordingly, the amount of each of the one or more ingredients delivered from the aerosol-generating material 38 to the user during the inhalation will also vary based on the amount of aerosol delivered to the user during the inhalation. Accordingly, the control circuitry is configured to use the inhalation detection signals received from the sensor 25 in order to determine an indication of an amount of an ingredient delivered from the aerosol-generating material to the user during the inhalation based on the inhalation detection signals.

In some embodiments, the control circuitry 28 is configured to determine a duration of the inhalation based on the inhalation detection signals received from the sensor 25, which can then be used in the determination of the indication of the amount of an ingredient delivered from the aerosol-generating material 38 to the user during the inhalation. In other words, the control circuitry 28 may be configured to determine the elapsed time for an inhalation based on the inhalation detection signals received from the sensor 25. As set out above, the sensor 25 may be configured to continuously or periodically output inhalation detection signals to the control circuitry, and the control circuitry is configured to use the change in these signals described above to determine a duration of an inhalation, for example by starting an inhalation timer when the inhalation detection signals change a first time and stop the inhalation timer when the inhalation detection signals change a second time. The control circuitry 28 may be configured to start the inhalation timer when the first non-zero inhalation detection signal is received, or when the first inhalation detection signal indicative of an inhalation being detected by the sensor 25 is received, such as at time 302 in FIGS. 3B and 3C respectively. The control circuitry 28 may then be configured to stop the inhalation timer when the next zero value inhalation detection signal is received, or when the next inhalation detection signal indicative of the sensor 25 no longer detecting an inhalation is received, such as at time 303 in FIGS. 3A-3C. Using FIGS. 3A-3C as an example, the duration of inhalation determined by the control circuitry 28 is the elapsed time between the time points 302 and 303.

Alternatively, the duration of an inhalation may be determined based on information contained within the inhalation detection signals, such as a time stamp associated with each inhalation detection signal. For example, in the case illustrated in FIG. 3B or 3C, the control circuitry 28 is configured to use the timestamp of a first non-zero inhalation detection signal (at time 302) received and the timestamp of the next zero value inhalation detection signal received (at time 303) to determine the duration of the inhalation. Alternatively, in the example illustrated in FIG. 3A, the control circuitry 28 is configured to use the timestamp of the first inhalation detection signal exceeding the detection threshold 301 received from the sensor 25, corresponding to time point 302 in the FIG. 3A, and the timestamp of the next inhalation detection signal indicative not exceeding the detection threshold received from the sensor, corresponding to time point 303 in FIG. 3A, to determine the duration of the inhalation.

In some embodiments, the control circuitry 28 is configured to determine a time between an inhalation and the next inhalation based on the inhalation detection signals. In other words, the control circuitry 28 is configured to determine the elapsed time between an inhalation and the inhalation that follows it. This can be achieved using the same techniques as described above with respect to determining the duration of an inhalation, such as using a timer, information contained within the inhalation detection signals or the period of output of the inhalation detection signals.

The control circuitry 28 may also be configured to determine one or more operational parameters of the aerosol provision system 10 which can be used in the determination of the indication of the amount of an ingredient delivered from the aerosol-generating material 38 to the user during the inhalation. For example, the operation parameter may be an amount of electrical power supplied to the aerosol generator 36 by the power source 14. The control circuitry 28 is then configured to determine the indication of the amount of the ingredient delivered from the aerosol-generating material 38 to the user during the inhalation based on an indication of the amount of electrical power supplied to the aerosol generator 36 by the power source 14 during the inhalation. For example, the indication of the amount of electrical power supplied could be the amount of power delivered or a voltage and/or current supplied to the aerosol generator 36 during the inhalation, or could be a power setting for the aerosol generator 36 during the inhalation, such as an integer between 1 and 10 or “1” for low, “2” for medium and “3” for high. The amount of aerosol generated by the aerosol generator 36 during an inhalation will vary depending on the amount of electrical power supplied to the aerosol generator 36, and therefore a more accurate determination of the indication of the amount of the ingredient delivered from the aerosol-generating material 38 to the user during the inhalation can be achieved by considered the amount of electrical power in the calculation.

Alternatively or in addition, control circuitry 28 may determine one or more other operational parameters of the aerosol provision system 10, such as an amount of charge in the power source 14, a temperature of the aerosol generator 36 or a temperature proximate to the aerosol generator 36, an amount and/or speed of airflow through the aerosol provision system 10, indications of which are then used to determine the indication of the amount of the ingredient delivered from the aerosol-generating material 38 to the user during the inhalation. The operational parameter may change or vary during an inhalation, for example a decrease in the amount of charge in the power source 14 or an increase in the temperature of the aerosol generator 36. The determination of the operational parameter by the control circuitry 28 may therefore correspond to a maximum value, a minimum value or an average, model or median value of the operational parameter during the inhalation. Equally, the indication of the operational parameter may represent one or more of a value for the operational parameter at the start of the inhalation, a value for the operational parameter at the end of the inhalation, a maximum value of the operation parameter during the inhalation, a minimum value of the operation parameter during the inhalation and an average, model and/or median value for the operational parameter during the inhalation.

The determination of the indication of the amount of the ingredient delivered from the aerosol-generating material 38 may also be based on other factors, such as a concentration of the ingredient in the aerosol-generating material 38. It will be appreciated that, for a given amount of aerosol generated by the aerosol generator 36 from the aerosol-generating material 38, the amount of the ingredient in the resulting aerosol will vary depending on the concentration, in other words the amount, of the ingredient in the aerosol-generating material 38. As described above, the ingredient may be an active substance, such as nicotine, caffeine, taurine, theine, vitamins such as B6 or B12 or C, melatonin, cannabinoids, or constituents, derivatives, or combinations thereof. The ingredient may be a flavorant, an aerosol-former material or a functional material such as a pH regulator, coloring agent, preservative, binder, filler, stabilizer or antioxidant. Accordingly, the concentration of the ingredient in the aerosol-generating material 38 can be considered when determining the indication of the amount of the ingredient delivered from the aerosol-generating material 38 in order to improve the accuracy of the determination. The concentration of the ingredient in the aerosol-generating material 38 may be provided to the control circuitry by the user, for example by inputting the concentration on a user input device associated with the aerosol provision system, or control circuitry 28 may be configured to determine the concentration of the ingredient in the aerosol-generating material 38, for example in response to the consumable 30 being attached to the aerosol provision system 10. The consumable 30 may comprise an electronic chip or tag, such as an RFIG tag, which the control circuitry 28 as able to read in order to determine the concentration of the ingredient in the aerosol-generating material 38, as well as other properties of the consumable 30, such as the identify of the manufacturer or the consumable, one or more flavorants or other ingredients contained within the aerosol-generating material 38 and the volume or mass of the aerosol-generating material 38 in the consumable.

As described above, the control circuitry 28 is configured to determine an indication of the amount of the ingredient delivered. The indication may represent the actual amount of the ingredient delivered, for example a mass or volume of the ingredient delivered from the aerosol-generating material 38 to the user during the inhalation. For example, control circuitry 28 may be configured to use an algorithm or look-up table to determine the amount of the ingredient delivered during the inhalation based on the inhalation detection signals. The algorithm or look-up table may be based on empirical data related to the aerosol provision system 10, such as the maximum or average mass flow of air through the air channel 23, or an amount of the ingredient delivered for a standard inhalation profile, such as 55 ml of air in a 3 second inhalation every thirty seconds (referred to as an 55/3/30 profile). If the amount of an ingredient delivered for a standard inhalation profile is known, then this can be scaled using a look-up table or an algorithm in order to determine the amount of the ingredient that is delivered for an inhalation with a different profile, such as the duration of the inhalation, the volume of aerosol delivered, the amount of electrical power delivered to the aerosol generator 36 and/or a temperature of the aerosol generator 36.

Alternatively, the indication of the amount of the ingredient delivered during the inhalation may relate to the amount of the ingredient delivered compared to a capacity of the aerosol-generating material storage area, such that the indication of the amount of the ingredient delivered indicates the amount of the ingredient and/or the aerosol-generating material remaining in the aerosol-generating material storage area. For example, the indication could be a percentage of the total amount of the aerosol-generating material present in the aerosol-generating material storage area when the aerosol-generating material storage area is full.

In some embodiments, the indication of the amount of the ingredient delivered during the inhalation is a rating on a fixed scale, for example an integer or real number between 0 and 10, where 0 is the lowest value and 10 is the highest value, although different forms and granulations of scales can also be used. In this case, an indication of 2 represents that a small amount of ingredient was delivered during the inhalation, whilst an indication of 10 represents that a maximum amount of the ingredient was delivered. This rating may be calculated by multiplying a duration of the inhalation by an indication of the operational parameter during the inhalation and applying one or more scaling factors, or by any other suitable calculation technique. Using such a rating on a scale allows for comparison between indications from different inhalations without requirement as exact or detailed a calculation as when the indication corresponds to the actual amount of the ingredient delivered.

The determination of the indication of the amount of the ingredient delivered from the aerosol-generating material 38 to the user may occur during the inhalation itself. In other words, the control circuitry 28 is configured to determine the indication of the amount of the ingredient that has been delivered as the inhalation takes place, such that the determination is ongoing during the inhalation. The determination of the indication of the amount of the ingredient delivered therefore occurs as inhalation detection signals are received from the sensor 25. For example, as described above, the control circuitry 28 may be configured to start the determination of the indication of the amount of the ingredient delivered in response to receiving inhalation detection signals from the sensor 25, or in response to a change in the inhalation detection signals received from the sensor 25. The determination of the indication of the amount of the ingredient delivered would then continue until the inhalation detection signals were no longer received from the sensor 25, or the inhalation detection signals received from the sensor 25 changed for a second time. Alternatively, the determination of the indication of the amount of the ingredient delivered could occur after the inhalation has concluded.

The indication of the amount of the ingredient delivered during an inhalation can be used to determine an indication of the amount of the ingredient delivered from the aerosol-generating material 38 to the user during each inhalation in a predetermined time period, such as a minute, an hour, a day, a week or longer. For example, the control circuitry 28 can be configured to determine an indication of the amount of the ingredient delivered during each inhalation in a rolling time period, which can then be summed to determine an indication of the total amount of the ingredient delivered from the aerosol-generating material 38 to the user in the rolling time period. As will be appreciated, a rolling time period is intended to mean the time immediately prior to any point in time, such that, for example, a rolling 24 hour period represents the 24 hour time period immediately prior to any point in time (in other words the most recent 24 hours in time at any point in time). Equally, the determination may be performed over a rolling time period of an hour, a day (24 hours), a week (7 days) or other period of time.

Additionally, the determination of the indication of the amount of the ingredient delivered from the aerosol-generating material 38 to the user during the predetermined time period may also be based on the time between each inhalation during the predetermined time period. For some ingredients, the amount of residual ingredient in the user's body system will decrease over time as the ingredient is absorbed, broken down, expelled or otherwise depleted from the user's body system. By considering the time between each inhalation and the duration of each inhalation in a predetermined time period, the indication of an amount of the ingredient delivered from the aerosol-generating material 38 to the user during the predetermined time period can indicate the amount of residual ingredient in the user's body system rather than the amount of the ingredient delivered to the user in the predetermined time period.

As will be appreciated, many users of aerosol provision systems 10 do not take single inhalations on the aerosol provision system 10, but rather perform a session on the aerosol provision system 10, where a session is a plurality of inhalations within a time period such as 1 to 2 minutes, sometimes longer such as 5 or 10 minutes. The control circuitry 28 can therefore be configured to determine an indication of the amount of the ingredient delivered from the aerosol-generating material 38 to the user over a plurality of inhalations, where a time between each of the plurality of inhalations is less than a predetermined time. The predetermined time may be set and altered by the user or the control circuitry 28, or may be a fixed value, for example based on empirical data. The predetermined time could be less than 1 minute, 1, 2, 5, 10 minutes or longer. The predetermined time can be defined as a rolling time period as described above such that each inhalation within the most recent predetermined time period is considered by the control circuitry to be part of the session. Alternatively, the predetermined time may be set such that the time between each inhalation must be less than the predetermined time in order for the inhalation to be considered by the control circuitry 28 as part of the same session. In this case, a session timer could be implemented to determine if an inhalation is part of a session, where the session timer is started when the inhalation detection signals indicate that an inhalation has been detected by the sensor, and the session timer is stopped when the duration between an inhalation and the next inhalation exceeds the predetermined time. Alternatively, as described above, a time stamp associated with each inhalation detection signal could be used to determine an indication of the amount of the ingredient delivered from the aerosol-generating material 38 to the user over the session. The determining of the indication of the amount of the ingredient delivered from the aerosol-generating material 38 to the user during the session may also be based on inhalation detection signals received for each inhalation during the session and the time in between each inhalation during the session. As described above, this determination may be performed for each inhalation in the session, for example a separate determination performed during each inhalation or after each inhalation has concluded in the session. Alternatively, the determination may be performed once, either during the session or after the entire session has concluded.

The control circuitry 28 is configured to compare the indication of the amount of the ingredient delivered to a threshold. The threshold may be set based on an amount of the ingredient delivered in a single puff (a puff threshold), for example to correspond to a safe usage limit of the ingredient and/or aerosol-generating material 38, or a safe usage limit of the aerosol provision system 10 for an inhalation, for example to prevent overheating or drying out of one or more of the components of the aerosol provision system 10. Alternatively, the threshold may correspond to a session threshold or a daily threshold, which may correspond to a safe usage limit of the ingredient and/or aerosol-generating material, or a safe usage limit of the aerosol provision system for a session and a 24 hour period respectively. While the above refers to a “safe” usage limit, it should be appreciated that this may not necessarily refer to a medically approved maximum dosage of the ingredient and/or aerosol generating material 38, but rather may refer to a recommended dosage somewhat below the medically approved maximum dosage, for example. Additionally, the threshold may be adjustable and/or set based by the user (either directly or automatically). Thus, more generally, the threshold represents an amount of the ingredient and/or aerosol generating material 38 over a given time period (e.g., an inhalation, 1 minute, 1 hour, etc.) which is typically below a medically approved maximum amount for the given time period.

As described above, the determination of the indication of an amount of an ingredient delivered may occur during the inhalation. In this case, the comparison between the indication of an amount of an ingredient delivered and the threshold may be performed during the inhalation, either continuously or periodically (such as every second or every 5 seconds). In other words, the indication of an amount of an ingredient delivered is constantly determined during the inhalation, and the value for the indication of an amount of an ingredient delivered at the current moment in time is compared to the threshold. Alternatively, comparing the indication of the amount of the ingredient delivered to the threshold can performed after each inhalation. In this case, the determination of indication of an amount of an ingredient delivered may occur either during the inhalation or after the inhalation.

The control circuitry 28 is configured provide a notification to the user based on the comparison between the indication of the amount of the ingredient delivered to the threshold. For example, a notification may be provided on the aerosol provision system 10, such as by activating an indicator light, emitting a sound from a speaker or displaying a message on a display screen on the aerosol provision device 20 and/or the consumable 30. The notification may also be a haptic notification on the aerosol provision system 10, such as a vibration or force feedback. For example, a vibration may be generated by an eccentric rotating mass (ERM) or piezoelectric actuator within the aerosol provision device 20 and/or the consumable 30, or a force may be generated by a motor within the aerosol provision device 20 and/or the consumable 30. The notification could also be a change in a mode of operation of the aerosol provision system 10 which the user would detect, such as switching off, disabling or otherwise preventing electrical power from being supplied to the aerosol generator 36. For example, the aerosol generator 36 could be disabled for a period of time, such as 5 seconds, 10 seconds, a minute or longer.

Alternatively, or in addition, the notification may be provided on an application on a remote device. For example, the user of the aerosol provision system 10 may have a remote device associated with, but separate from, the aerosol provision system 10, and the control circuitry 28 is configured to communicate with the remote device, for example by Bluetooth, Bluetooth Low Energy (BLE), ANT+, Wi-Fi or any other suitable wireless communication method. The control circuitry 28 can be configured to communicate with the remote device such that the notification is provided to the user on the remote device, such as on an application installed on the remote device. For example, a message may be displayed on a display screen on the remote device, an indicator light activated, a sound emitted from a speaker or a haptic notification means on the remote device as described above. The remote device may include any suitable electronic device that can be communicatively coupled to the aerosol provision system 10. For example, the remote device may include a mobile device (such as a smartphone), a PDA, a personal computer, laptop, tablet, smartwatch, etc.

Further, one or more parameters associated with the notification may be adjustable by the user. For example, the user may be able to adjust the number, brightness and/or color of the indictor light that is activated, the volume, pitch and or duration of the sound emitted and/or the message that is displayed. The user may also be able to adjust one or more parameters of the haptic notification. For example the user may be able to adjust the duration, magnitude and/or pattern of the vibrations or forces provided by the actuator and motor respectively.

The notification may be configurable by the user. In other words, the user may be able to adjust the one or more parameters associated with the notification on the aerosol provision system 10 and/or the remote device regardless of whether the notification is provided on the aerosol provision system 10 or the remote device. For example, the user may be able to use the application on the remote device to adjust one or more of the parameters associated with the notification even though the notification itself is provided on the aerosol provision system 10. For example, the user may disable notifications during an inhalation such that notifications are only received when an inhalation is not detected by the sensor 25.

In some embodiments, the control circuitry 28 is configured to provide the notification to the user when the indication of an amount of an ingredient delivered from the aerosol-generating material to the user during the inhalation exceeds the threshold. The indication of the amount of the ingredient delivered from the aerosol-generating material to the user during the inhalation might represent a proportion or percentage of the amount of the ingredient delivered from the aerosol-generating material to the user during the inhalation compared to the threshold. For example, the indication might be a percentage of the threshold, such as 10%, 20%, 50%, 80% or 110%, and the notification is provided to the user when the percentage is greater that 100%. In the embodiments where the comparison between the indication of the amount of the ingredient delivered and the threshold occurs during the inhalation, the notification can be provided to the user during the inhalation as soon as the amount of the ingredient delivered from the aerosol-generating material to the user exceeds the puff threshold.

Alternatively, the notification could be provided to the user when the indication of the amount of the ingredient delivered is within a range of the threshold, such as 50%, 75% or 90% of the threshold. This ensures that the user provided with the notification before the threshold is exceeded. In this case, the notification could indicate to the user how close to the threshold the indication of an amount of an ingredient delivered is, for example by displaying the ratio of the values or by a visual means such as a progress bar or by lighting a particular number of indicator lights on the aerosol provision system or the remote device.

In some embodiments, the aerosol provision system 10 is configured to display, or otherwise communicate, the indication of the amount of the ingredient delivered to the user. For instance, the aforementioned progress bar or the like could be displayed to the user. This may be displayed at all times, or in response to a user request for this information. However, when the indication of the amount of the ingredient delivered is within a range of the threshold, or exceeds the threshold, the control circuitry 28 provides the notification to the user. For instance, when the progress bar fills to 90%, with 100% representing the threshold, the control circuitry 28 may provide the notification to the user, which may for example, be a change in the display (e.g., the color of the progress bar may change) or a haptic or audible notification may be provided.

In some embodiments, the control circuitry 28 is configured to determine default user behavior based on the indication of the amount of the ingredient delivered from the aerosol-generating material 38 to the user. In other words, the control circuitry 28 is configured to detect patterns in one or more inhalations by the user based on the indication of the amount of the ingredient delivered from the aerosol-generating material 38 to the user. The default user behavior can also be determined from the data determined by the control circuitry 28 for one or more inhalations which is used to generate the indication of the amount of the ingredient delivered, such as the duration of an inhalation, the duration of a session, a time between inhalations, the amount of electrical power delivered to the aerosol generator 36 during the inhalation, a power level or setting for the aerosol generator 36 for the inhalation and the type and/or concentration of one or more of the ingredients in the aerosol-generating material 38. These patterns are then used to default user behavior with respect to the amount of the ingredient delivered from the aerosol-generating material 38 to the user during an inhalation.

The default user behavior can be determined over an inhalation, such that the default user behavior relates to a typical inhalation performed by the user on the aerosol provision system 10. For example, when the user first uses the aerosol provision system 10, in other words where only a single inhalation has been detected, the default user behavior may correspond to the indication of the amount of the ingredient delivered for that inhalation. As the user performs more inhalations on the aerosol provision system 10, the default user behavior can be updated based on the indication of the amount of the ingredient delivered for each subsequent inhalation, thereby refining the typical inhalation performed by the user on the aerosol provision system 10.

The indication of the of the amount of the ingredient delivered for each inhalation can also be used to determine default user behavior over a session; in other words, the default user behavior is determined over a plurality of inhalations, where a time between each of the plurality of inhalations is less than a predetermined time as described above. The indication of the amount of the ingredient delivered for each inhalation can also be used to determine default user behavior over another period, such as a rolling time period or a rolling 24 hour period as described above, a week, a month and/or a year.

Additionally, the control circuitry 28 can continually update the determined default user behavior based on changes in the indication of the amount of the ingredient delivered from the aerosol-generating material 38 to the user for inhalations over time. For example, where the control circuitry 28 is configured to determine a time between an inhalation and the next inhalation based on the inhalation detection signals, the default user behavior can also be determined based on the time between the inhalation and the next inhalation. This allows patterns of behavior to be detected for the user, such as if the user takes a series of puffs, such as a session described above, then has an extended period between sessions, such as 30 minutes, 1 hour or longer, or whether the user takes a small number of inhalations, such as 1 or 2, but spaced more regularly, such as every 10 of 20 minutes. Equally, the data collected may allows the control circuitry 28 to determine particular times of the day when the user takes more inhalations, such as in the mornings or the evenings, or if the number and duration of inhalations in a session change during a day. For example, the user may have a session comprising a plurality of long inhalations with a high power setting in the morning, but sessions in the evening comprise fewer, short inhalations with a lower power setting. The user may perform more inhalations on weekdays, whilst over the course of a month or a year the data may indicate that the user is performing fewer inhalations, for example due to the user trying to cut down their usage of the aerosol provision system. Such default behavior can be determined based on the indication of the amount of the ingredient delivered from the aerosol-generating material 38 to the user for a plurality of inhalations.

As described above, for some ingredients the amount of residual ingredient in the user's body system will decrease over time as the ingredient is absorbed, broken down, expelled or otherwise depleted from the user's body system. The indication of an amount of the ingredient delivered from the aerosol-generating material 38 to the user can indicate the amount of residual ingredient in the user's body system rather than the amount of the ingredient delivered to the user in the predetermined time period, and therefore the default user behavior can also be based on the amount of residual ingredient in the user's body system.

The control circuitry 28 may be configured to determine or alter the threshold described above based on the default user behavior, thereby personalizing or otherwise tailoring the operation of the aerosol provision system 10 to the user. Alternatively or in addition, the user may be able to alter one or more of the thresholds, for example by providing an input on an input device on the aerosol provision device 20 or consumable 30, or through an application on an associated remote device, thereby giving the user additional control over the operation of the aerosol provision system 10.

The control circuitry 28 may also be configured to alter a mode of operation of the aerosol generation system 10 based on the default user behavior, such as an amount of electrical power supplied to the aerosol generator 36 by the power source 14, the temperature of the aerosol generator 36, a sensitivity or detection threshold on the sensor 25, the color and/or number of light indicators illuminated and/or the volume, pitch and/or duration of a sound emitted on the aerosol provision device 20 for an inhalation.

For example, if it is determined that the user takes longs inhalations, such as greater than 10 seconds, the control circuitry 28 can be configured to alter the amount of electrical power supplied to the aerosol generator 36 by the power source 14 during the inhalation in order to prevent dry out or overheating of the aerosol generator 36. The power supplied to the aerosol generator 36 may be set to an initial value or power setting, and then reduced as the inhalation continues. Alternatively, if is determined that the user takes very small or gentle inhalations, for example with a low air speed or mass flow, the control circuitry 28 can be configured to change a sensitivity or detection threshold on the sensor 25 to ensure that an inhalation is properly detected for the user.

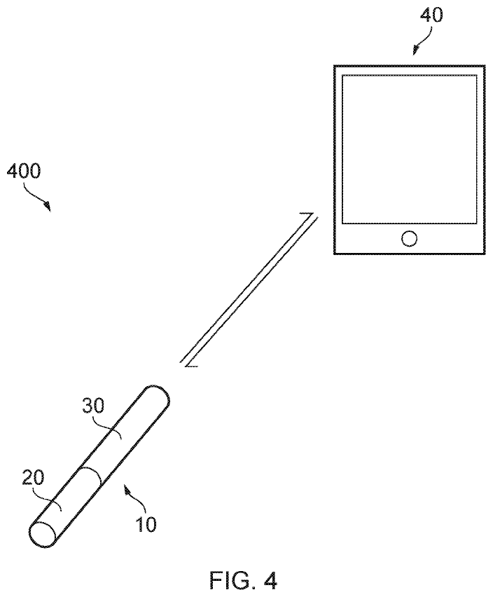

FIG. 4 illustrates a system 400 comprising an aerosol provision system 10 configured to generate aerosol from an aerosol-generating material 38, such as described above. The system 400 also comprises a computer 40 configured to receive inhalation detection signals from a sensor 25 configured to determine an indication of an amount of an ingredient delivered from the aerosol-generating material 38 aerosolized by an aerosol generator 36 to the user during the inhalation based on inhalation detection signals received from a sensor 25 of the aerosol provision system 10. The computer 40 is also configured to compare the indication of the amount of the ingredient to a threshold and provide a notification to the user based on the comparison between the indication of the amount of the ingredient delivered to the threshold.

As described above and illustrated in FIG. 4, the computer 40 may be a remote device associated with the user and in communication with the aerosol provision system 10. Accordingly, it will be appreciated that the functions of the control circuitry described herein, such determining an indication of an amount of an ingredient delivered, determining default user behavior, comparing the indication of the amount of the ingredient to a threshold and providing a notification to the user may be performed by a computer 40 separate from the aerosol provision system 10, such as a remote device.

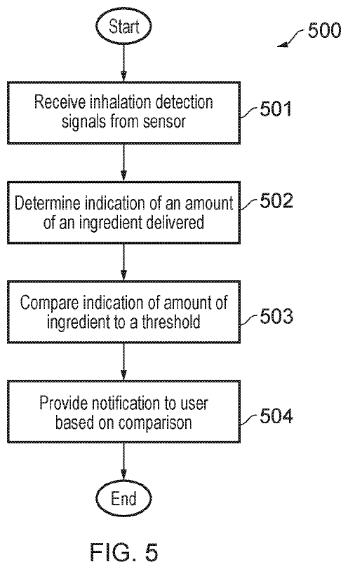

FIG. 5 is a flow chart of a method 500 of determining an amount of an ingredient delivered to a user of an aerosol provision system 10. The method begins at 501, where inhalation detection signals are received from a sensor configured to detect an inhalation on the aerosol provision system by a user of aerosol provision system. At 502, an indication of an amount of an ingredient delivered from an aerosol-generating material aerosolized by an aerosol generator to the user during the inhalation is determined based on the inhalation detection signals. At 503 the indication of the amount of the ingredient is compared to a threshold. At 504 a notification is provided to the user based on the comparison between the indication of the amount of the ingredient delivered to the threshold.

The method 500 illustrated in FIG. 5 may be stored as instructions on a computer readable storage medium, such that when the instructions are executed by a processor, the method 500 described above is performed. The computer readable storage medium may be non-transitory.

Although it has been described above that the indication of the amount of the ingredient delivered is compared to a threshold, and subsequently a notification to the user is provided based on the comparison between the indication of the amount of the ingredient delivered to the threshold, it should be appreciated that in some implementations a plurality of thresholds (e.g. two) may be provided, and the control circuitry 28 is configured to compare the indication of the amount of the ingredient delivered to each of the thresholds. For example, if the amount of ingredient delivered exceeds a first amount, a notification may be provided alerting the user that a first level of ingredient has been delivered. Continued use may cause the amount of ingredient delivered to exceed a second amount, at which point a subsequent notification may be provided alerting the user that a second level of ingredient has been delivered. Alternatively, the first and second thresholds may define a range for the amount of ingredient delivered between which no notification is issued. If the amount of ingredient delivered is above or below the range, then the control circuity 28 may provide a notification accordingly. In the context of the lower threshold, this may be set by a user in accordance with a nicotine cessation program or the like, and as such the notification may provide the user with a message stating the amount of ingredient delivered is below the threshold. The notification and/or action taken by the control circuitry 28 may be the same or different for the comparison of the ingredient delivered compared to the different thresholds.

As described above, the present disclosure relates to (but it not limited to) an aerosol provision system comprising an aerosol generator configured to aerosolize an aerosol-generating material. The aerosol provision system also comprises a sensor configured to detect an inhalation on the aerosol provision system by a user of the aerosol provision system, and output corresponding inhalation detection signals to control circuitry. The control circuitry is configured to determine an indication of an amount of an ingredient delivered from the aerosol generating material to the user during the inhalation based on the inhalation detection signals, compare the indication of the amount of the ingredient delivered to a threshold, and provide a notification to the user based on the comparison between the indication of the amount of the ingredient delivered to the threshold.

Thus, there has been described an aerosol provision system, a system comprising an aerosol provision system and a computer, a method of operating an aerosol provision system, and a computer readable storage medium.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the disclosure as defined by the claims or limitations on equivalents to the claims, and that other embodiments may be utilized and modifications may be made without departing from the scope of the claims. Various embodiments of the disclosure may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

1. An aerosol provision system comprising:
an aerosol generator configured to aerosolize an aerosol-generating material; and
a sensor configured to detect an inhalation on the aerosol provision system by a user of the aerosol provision system, and output corresponding inhalation detection signals to control circuitry, wherein the control circuitry is configured to:
determine an indication of an amount of an ingredient delivered from the aerosol-generating material to the user during the inhalation based on the inhalation detection signals;
compare the indication of the amount of the ingredient delivered to a threshold; and
provide a notification to the user based on the comparison between the indication of the amount of the ingredient delivered to the threshold.

2. The aerosol provision system of claim 1, wherein comparing the indication of the amount of the ingredient delivered to the threshold is performed based on an indication of the amount of the ingredient delivered from the aerosol-generating material to the user during each inhalation in a predetermined period.

3. The aerosol provision system of claim 2, wherein the predetermined period is an hour, 24 hours or a week.

4. The aerosol provision system of claim 1, wherein comparing the indication of the amount of the ingredient delivered to the threshold is performed after each inhalation.

5. The aerosol provision system of claim 1, wherein comparing the indication of the amount of the ingredient delivered to the threshold is performed during the inhalation.

6. The aerosol provision system of claim 5, wherein comparing the indication of the amount of the ingredient delivered to the threshold is performed at a predetermined interval during the inhalation.

7. The aerosol provision system of claim 1, wherein the determination of the indication of the amount of the ingredient delivered from the aerosol-generating material to the user is further based on a concentration of the ingredient in the aerosol-generating material.

8. The aerosol provision system of claim 1, wherein the ingredient is nicotine, caffeine, taurine, theine, a vitamin, melatonin, or a cannabinoid.

9. The aerosol provision system of claim 1, wherein the notification is provided to the user when the indication of the amount of the ingredient delivered exceeds the threshold.

10. The aerosol provision system of claim 1, wherein the control circuitry is configured to determine default user behavior based on the indication of the amount of the ingredient delivered from the aerosol-generating material to the user.

11. The aerosol provision system of claim 10, wherein the default user behavior is determined over an inhalation.

12. The aerosol provision system of claim 10, wherein the control circuitry is configured to determine a time between the inhalation and a next inhalation based on the inhalation detection signals, and wherein determining default user behavior is also based on the time between the inhalation and the next inhalation.

13. The aerosol provision system of claim 10, wherein the default user behavior is determined over a plurality of inhalations, and a time between each of the plurality of inhalations is less than a predetermined time.

14. The aerosol provision system of claim 10, wherein the default user behavior is determined over a rolling time period.

15. The aerosol provision system claim 10, where the threshold is determined based on the determined default user behavior.

16. The aerosol provision system of claim 1, wherein the notification is provided on the aerosol provision system.

17. The aerosol provision system of claim 1, wherein the notification is provided on an application on a remote device.

18. The aerosol provision system of claim 1, wherein the notification is a haptic notification.

19. The aerosol provision system of claim 18, wherein a parameter of the haptic notification is adjustable by the user of the aerosol provision system.

20. The aerosol provision system of claim 1, wherein the notification is configurable by the user.

21. The aerosol provision system of claim 1, wherein the notification is provided to the user based on the comparison between the indication of the amount of the ingredient delivered to a plurality of thresholds.

22. A system comprising:
an aerosol provision system configured to generate aerosol from an aerosol-generating material; and
a computer configured to:
determine an indication of an amount of an ingredient delivered from the aerosol-generating material aerosolized by an aerosol generator to the user during the inhalation based on inhalation detection signals received from a sensor of the aerosol provision system;
compare the indication of the amount of the ingredient to a threshold; and
provide a notification to the user based on the comparison between the indication of the amount of the ingredient delivered to the threshold.

23. A method of operating an aerosol provision system comprising:
receiving inhalation detection signals from a sensor configured to detect an inhalation on the aerosol provision system by a user of aerosol provision system;
determining an indication of an amount of an ingredient delivered from an aerosol-generating material aerosolized by an aerosol generator to the user during the inhalation based on the inhalation detection signals;
comparing the indication of the amount of the ingredient to a threshold; and
providing a notification to the user based on the comparison between the indication of the amount of the ingredient delivered to the threshold.

24. A non-transitory computer readable storage medium comprising instructions which, when executed by a processor, cause the processor to perform a method comprising:
receiving inhalation detection signals from a sensor configured to detect an inhalation on the aerosol provision system by a user of aerosol provision system;
determining an indication of an amount of an ingredient delivered from an aerosol-generating material aerosolized by an aerosol generator to the user during the inhalation based on the inhalation detection signals;
comparing the indication of the amount of the ingredient to a threshold; and
providing a notification to the user based on the comparison between the indication of the amount of the ingredient delivered to the threshold.
